(19) United States
(12) Patent Application Publication
(10) Pub. No.: US 2004/0112388 A1
(43) Pub. Date: Jun. 17, 2004

(54) Laryngeal masks

(76) Inventor: Jeremy Colin Russell, Hythe (GB)

Correspondence Address: LOUIS WOO, LAW OFFICE OF LOUIS WOO, 717 NORTH FAYETTE STREET, ALEXANDRIA, VA, 22314, US

(21) Appl. No.: 10/623,493

(22) Filed: Jul. 22, 2003

(30) Foreign Application Priority Data
Aug. 10, 2002 (GB) 0218631.0

(51) Int. Cl. A62B 9/06; A61M 16/00

(52) U.S. Cl. 128/207.14; 128/207.15

(57) ABSTRACT

A laryngeal mask airway has a tubular shaft with a mask portion secured at its patient end. The mask portion comprises a PVC mount and an inflatable cuff secured around an edge of the mount. The posterior surface of the mount and cuff has a coating of a hydrophilic polymer or copolymer which becomes slippery when hydrated to ease insertion of the airway into the patient.

### BACKGROUND OF THE INVENTION

This invention relates to laryngeal masks.

Laryngeal mask airways are used to ventilate and to supply anaesthetic gas to a patient during surgery. Laryngeal mask airways differ from endotracheal tubes, which extend into the trachea and terminate beyond the vocal folds. By contrast, laryngeal mask airways have a tubular shaft opening into the centre of a generally elliptical mask or cuff, which is inflated to seal in the region of the hypopharnyx, at the top of the trachea. The cuff is inflated with air supplied along a small-bore inflation line communicating with the interior of the cuff. Laryngeal masks are described in, for example: U.S. Pat. No. 5,355,879, U.S. Pat. No. 5,305,743, U.S. Pat. No. 5,297,547, U.S. Pat. No. 5,282,464, GB 2267034, U.S. Pat. No. 5,249,571, U.S. Pat. No. 5,241,956, U.S. Pat. No. 5,303,697, GB 2317830, GB 2249959, GB 2111394, EP 448878, U.S. Pat. No. 4,995,388, GB 2205499, GB 2128561, GB 2298797, GB 2321854, GB 2334215, GB 2323289, GB 2323290, GB 2318735, GB 2330312, WO 01/13980, EP 1207927, GB 2337020, GB 2334215, GB 2331932, GB 2354950, GB 2359996, GB 0201958.6, GB 0201094.0 and GB 0127184.0.

Because the patient end of the laryngeal mask is relatively large and the rear surface of the mask needs to be slid along the posterior wall of the pharynx for introduction it is usual to lubricate the mask in some way. Where the mask is of silicone rubber it is often lubricated by spraying it with water, which wets the material to make it slippery. Where the mask is of PVC and other materials it may be lubricated by smearing a lubricating jelly over the surface of the mask just before insertion.

### BRIEF SUMMARY OF THE INVENTION

It is an object of the present invention to provide an alternative laryngeal mask.

According to the present invention there is provided a laryngeal mask airway having a mask portion at least of a polymeric material, at least the posterior surface at the patient end of the mask portion being coated with a hydrophilic material such that the posterior surface at the patient end becomes slippery when wetted, either in the patient or after treatment with an aqueous medium.

The mask portion is preferably of PVC and the hydrophilic material is preferably of a polymer or copolymer. The hydrophilic material may be selected from a group comprising polyvinylpyrolidenes, polyurethanes, polyvinyl alcohol and polyethylene glycol.

A laryngeal mask airway according to the present invention, will now be described, by way of example, with reference to the accompanying drawings.

### BRIEF DESCRIPTION OF THE DRAWINGS

FIG. 1 is a perspective view of the airway; and

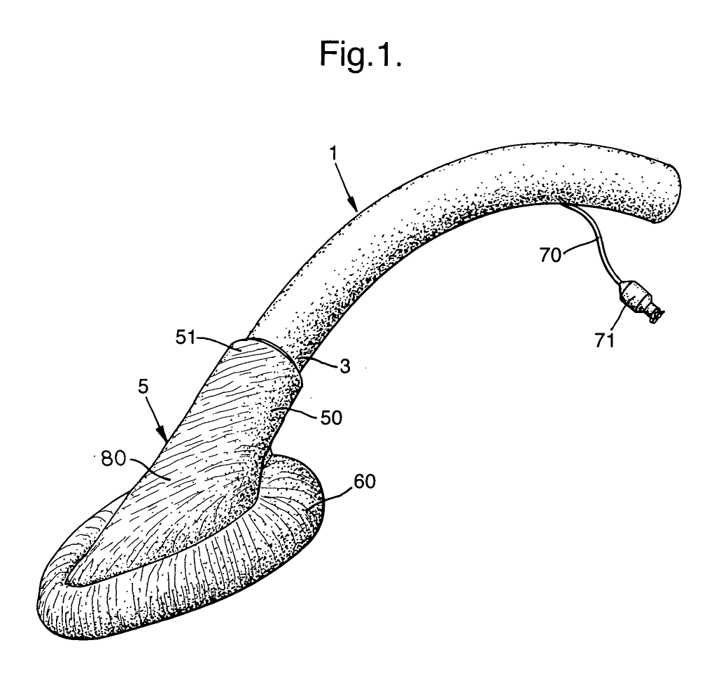

FIG. 2 is a sectional side elevation view of the airway;

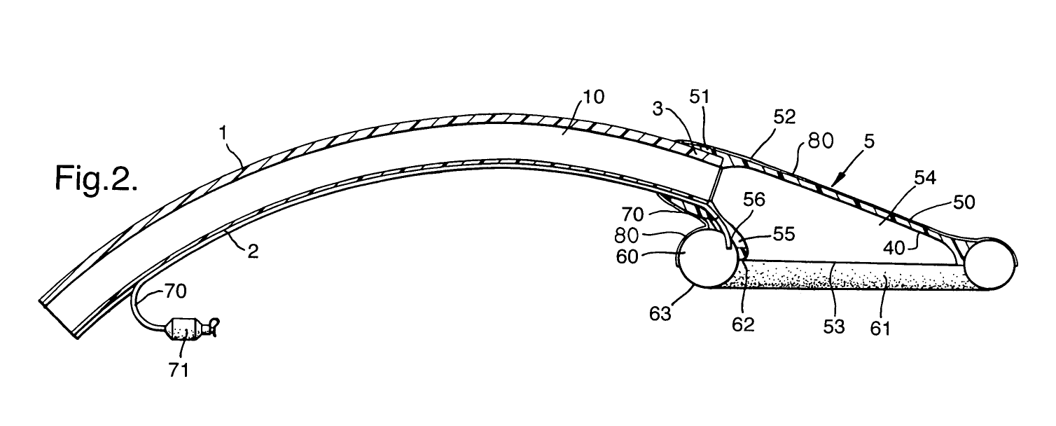

### DETAILED DESCRIPTION OF PREFERRED EMBODIMENT

The airway includes a curved tube or shaft 1 of PVC having a channel 2 in the form of a groove extending along its length on its outside surface and on the inside of its curve. The shaft 1 is preferably made by extrusion or moulding and may be reinforced by means of an embedded helical element, such as of metal or plastics. At its patient end 3, the shaft 1 is attached to a mask portion 5.

The mask portion 5 comprises a mount member 50 of a relatively stiff but compliant PVC and an inflatable cuff 60, also of PVC, attached to the mount member. The mount member 50 is hollow and of generally shoe shape, having a tubular extension or collar 51 at its upper or posterior side located at the rear, left-hand or machine end of the mount. The patient end 3 of the shaft 1 is bonded into one end of the collar 51. The other end of the collar 51 opens into a central recess or atrium 54 within the mount 50. The internal, anterior surface of the roof 40 of the atrium 54 is arched transversely but is substantially straight, or is slightly concave, along its longitudinal centre line. The roof 40 is uninterrupted by any surface projections or formations that would impede free movement of the epiglottis over the roof. Viewed in plan, the mount 50 is oval with its lower or anterior side 53 lying on a flat plane extending at an angle of about 30° to the axis of the patient end 3 of the collar 51. A channel 55 in the form of a groove extends along the inside of the mount member 50 in line with the groove 2 along the shaft 1 and this opens through a hole 56 into the cuff 60.

The cuff 60 may be of any conventional form, such as described in GB 2323291 or GB 2321854. The cuff 60 is only shown schematically in the drawings but is of annular, elliptical shape, being attached to the forward end surface 53 of the mount member 50 and having a central opening 61 into the atrium 54. The cuff 60 is of a thin, flexible plastics material so that it can be deflated to a low profile for insertion and can be inflated to seal with surrounding tissue when correctly positioned.

In general, the patient end of the tubular portion 1 is located to the rear of the rear side 62 of the opening 61, that is, on the side towards the machine end of the airway, and is preferably located approximately midway across the width of the sealing cuff. Instead of the tube and mount being separate components, they could be provided by one integral moulded component, with the location where the tubular portion increases in internal diameter being regarded as the patient end of the tubular portion.

The cuff 60 is inflated and deflated by means of an inflation line 70 in the form of a small-diameter flexible plastics tube extending along the groove 2 in the shaft 1.

As so far described, the laryngeal mask airway is conventional. The mask also includes a coating 80 of a solid hydrophilic material such as polyvinylpyrolidenes, polyurethanes, polyvinyl alcohol, polyethylene glycol and other polymers and copolymers. The coating 80 extends over at least the posterior surface of the airway at its forward end. In particular, the coating 80 extends over the posterior surface of the mount 50 and the cuff 60, that is, the surfaces that contact the posterior wall of the pharnyx during insertion. The coating could, instead, be applied to the entire airway. The coating is applied to the airway during manufacture so that the airway is supplied coated to the user in a dry form. Just before use, the user sprays the patient end of the airway with water so that the coating 80 is wetted and becomes slippery or lubricious. This allows the airway to be slid easily into position without the need to use a lubricating jelly. It may not be necessary to wet the airway before insertion because the coating will become hydrated by moisture in the patient.

It will be appreciated that various different forms of coating could be used. The coating could be applied to the entire surface of the airway although it is only necessary to apply the coating to those surface that slide over patient tissue during insertion. The shaft and mask portion need not be of PVC but could be of other polymeric materials.

What I claim is:

1. A laryngeal mask airway comprising a tubular portion and a mask portion at a patient end of said tubular portion, said mask portion being of a polymeric material, wherein at least a posterior surface at a patient end of said mask portion is coated with a hydrophilic material such that said posterior surface becomes slippery when wetted, either in the patient or after treatment with an aqueous medium.

2. A laryngeal mask according to claim 1, wherein said mask portion is of PVC.

3. A laryngeal mask according to claim 1, wherein said hydrophilic material is of a polymer or copolymer.

4. A laryngeal mask according to claim 1, wherein said hydrophilic material is selected from a group comprising polyvinylpyrolidenes, polyurethanes, polyvinyl alcohol and polyethylene glycol.

5. A laryngeal mask airway comprising: a tubular portion; a mount of PVC secured with a patient end of said tubular portion; and an inflatable cuff secured with said mount, wherein at least a posterior surface at a patient end of said mount is coated with a hydrophilic polymer or copolymer such that said posterior surface becomes slippery when wetted, either in the patient or after treatment with an aqueous medium.
